(19) United States
(12) Patent Application Publication
(10) Pub. No.: US 2015/0107627 A1
(43) Pub. Date: Apr. 23, 2015

(54) Self-Washing Closable Surgical Case Cart Apparatus, System, and Method

(71) Applicants: Barry Miles Snyder, Valrico, FL (US); Travis Lee White, Brandon, FL (US); Jeffrey Scott Ashcraft, Endicott, NY (US)

(72) Inventors: Barry Miles Snyder, Valrico, FL (US); Travis Lee White, Brandon, FL (US); Jeffrey Scott Ashcraft, Endicott, NY (US)

(21) Appl. No.: 14/056,945

(22) Filed: Oct. 18, 2013

(51) Int. Cl. A61B 19/02; B60B 33/00; A47B 67/04; A47B 81/00; A61B 19/00; A47B 67/02

(52) U.S. Cl. CPC A61B 19/0248; A61B 19/34; A47B 67/02; A47B 67/04; A47B 81/00; B60B 33/0036; A61B 2019/025; A61B 2019/0249
USPC 134/22.1; 312/229; 312/209

(57) ABSTRACT

Disclosed is a self-washing closable surgical case cart apparatus, system and method. The self-washing closable surgical case cart apparatus incorporates a 360-degree universal fluid delivery system, compatible with external sources for fluid monitoring and delivery, that assures all surfaces therein may be decontaminated inexpensively, quickly, completely, and efficiently. In addition to its self-cleaning features, the design provides improved ergonomics for users as well as enhanced protection from floor-level contaminants. Methods for decontamination are also presented that take advantage of its unique features.

### CROSS-REFERENCES TO RELATED APPLICATIONS

Not applicable.

### STATEMENT REGARDING FEDERALLY SPONSORED RESEARCH OR DEVELOPMENT

Not applicable.

### MICROFICHE APPENDIX

Not applicable.

### BACKGROUND OF THE INVENTION

1. Field of the Invention

This invention relates to the field of surgical case cart apparatus, systems and methods for decontaminating the same.

2. Description of Related Art

Designed for the orderly storage of instruments and supplies for medical operations, surgical case carts have become a necessary investment for any hospital, clinic, or private practice of medicine. The utilization of well-designed surgical case carts can help keep track of inventory levels and ensure that medical staff enjoys easy access to all necessary procedural instruments and supplies. Benefits include efficient use of space, effective time management, economically appropriate use of staff, prevention of infection and the accurate capture of information.

Typically, surgical case carts are constructed of welded stainless steel and utilize casters for portability and handles for maneuverability. In closable surgical case cart designs, latchable doors with seals provide access to and contamination protection for the interior of the cart, into which one or more shelves may be introduced for the placement of sterile supplies, implants, and surgical tools to be conveyed to and from surgery. They provide greater security for sterile contents, and without a dedicated clean pathway from the point at which they are stocked with sterile supplies to the operating room or other point of use, they become a necessity.

Repeatedly decontaminating and drying surgical case carts is crucial to their effective use. Reducing the time it takes to clean up after one procedure and prepare for the next—i.e., turnover time—is a critical exercise for any surgical department. The easier a surgical case cart is to clean and dry, the quicker it can be put back into service.

Conventional closable surgical case carts may be cleaned by hand, a labor-intensive, inefficient, and time-consuming task. Alternatively, a case cart washing system may be employed into which such a surgical case cart is inserted with the doors open so that the cleaning jets of the system may reach the inside of the cabinet to decontaminate it, but such systems represent a substantial capital investment for any hospital, clinic, or private medical practice, and require ongoing maintenance and repair, which can add to operating costs. For this reason, only a small percentage of hospitals and other medical facilities employ case cart washing systems.

### BRIEF SUMMARY OF THE INVENTION

The present invention is a self-washing closable surgical case cart system and method of decontaminating the same inexpensively, quickly, completely, and efficiently. It comprises a closable surgical case cart incorporating a 360-degree universal fluid delivery system, compatible with external sources for fluid monitoring and delivery, that assures all surfaces within the closable surgical case cart may be decontaminated. Fluid removal may be accomplished through a pluggable drain and a pitched floor allowing for enhanced water removal. The drain may be accessed via an outlet coupler, which may be a universal hose hookup. to allow drainage to a receptacle via a hose or other suitable fluid drainage channel, or free drainage without a hose.

In addition to these self-cleaning features, the design of the instant invention provides improved ergonomics for users as well as enhanced protection from floor-level contaminants. The lowest position for a shelf within the closable surgical case cart is positioned a minimum of 18 to 24 inches above the floor, reducing bending and lifting when loading and unloading, and creating greater distance from potential contamination from the floor. The wheels of the closable surgical case cart may be larger than those commonly used in other designs to allow for easier maneuverability, and directional or rigid front wheels aid in steering while the closable surgical case cart is in transit. Moreover, the height of the closable surgical case cart may be made adjustable for the convenience and comfort of the user.

A method of decontaminating the closable surgical case cart, taking advantage of its unique self-washing features, is also disclosed. This method broadly includes connecting a decontaminating fluid source to the closable surgical case cart, introducing the decontamination fluid or fluids into the closable surgical case cart through the 360-degree universal fluid delivery system, disconnecting the decontaminating fluid source, and allowing the closable surgical case cart to air dry or towel drying it prior to placing it into service.

### BRIEF DESCRIPTION OF THE SEVERAL VIEWS OF THE DRAWINGS

FIG. 1 is a front left isometric view of one embodiment of a closable surgical case cart, showing a coupler window and a ball tank washer.

FIG. 2 presents a front right isometric view of this particular embodiment of a closable surgical case cart revealing a hidden coupler supply line to a ball tank washer.

FIG. 3 provides a front view of this particular embodiment of a closable surgical case cart, adjusted to its lowest position.

FIG. 4 is another front view of this particular embodiment of a closable surgical case cart, adjusted to its highest position.

### REFERENCE NUMERALS IN THE DRAWINGS

2 Self-Washing Closable Surgical Case Cart Apparatus
4 Cabinet
5 Channel Support
6 Lower Assembly
7 Side
8 Door Clip
10 Roof
12 Cabinet Doors
14 Door Latch
16 Hinge
18 Handle
20 Fixed Wheel
22 Directional Wheel
24 Castor Brake
26 Frame
28 Solid Shelf
30 Grated Self
32 Inlet Coupler
33 Fluid Supply Lines
34 Ball Tank Washer
35 Interior Shelf
36 Adjustable Shelf Supports
38 Pitched Floor
40 Pluggable Drain
42 Wheel Lock
44 Lower Nut
46 Upper Nut
48 Threaded Shaft
50 Outlet Coupler
80 Back

### DETAILED DESCRIPTION OF THE INVENTION

A self-washing closable surgical case cart apparatus, system and method of decontaminating the same inexpensively, quickly, completely, and efficiently is disclosed herein. The system comprises a self-washing closable surgical case cart apparatus 2 incorporating a 360-degree universal fluid delivery system, compatible with external sources for fluid monitoring and delivery, that assures all surfaces within the closable surgical case cart apparatus 2 may be decontaminated. Fluid removal may be accomplished through a pluggable drain 40 and a pitched floor 38 allowing for enhanced water removal. The drain 40 may be accessed via an outlet coupler 50, which may be a universal hose hookup to allow drainage to a receptacle through a hose, or free drainage without a hose.

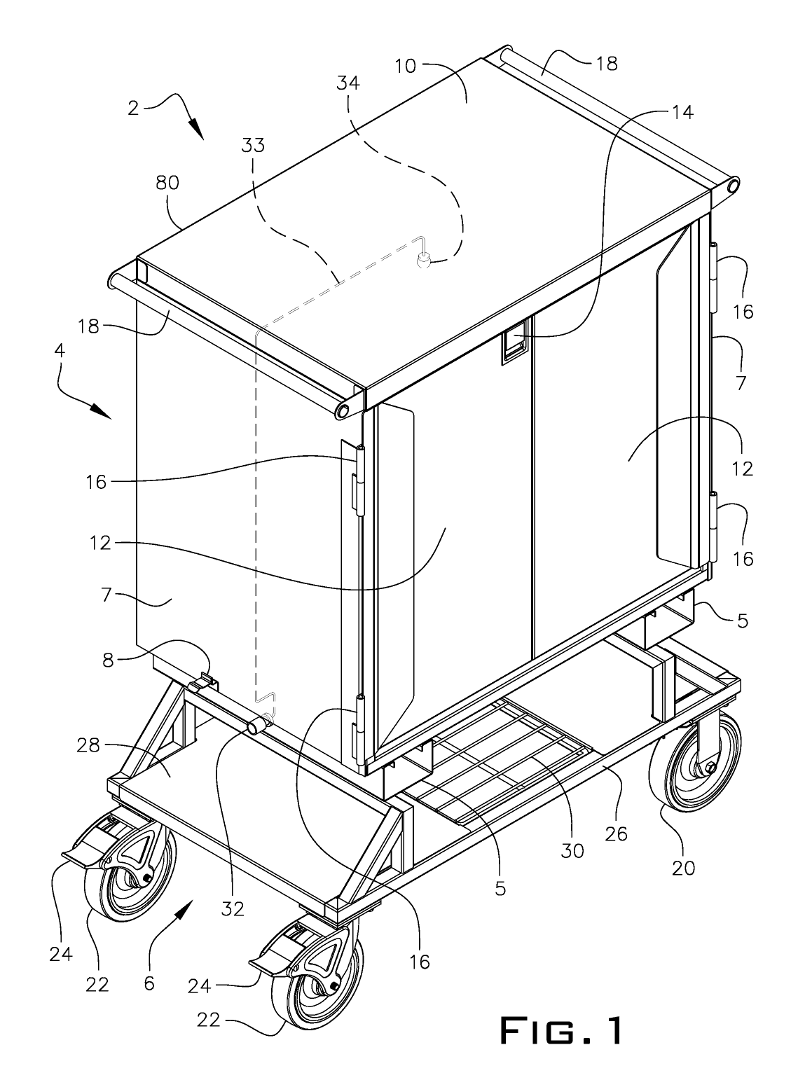

As illustrated in FIG. 1, the self-washing closable surgical case cart apparatus 2 is comprised of a cabinet 4, which may incorporate one or more channel supports 5, and a lower assembly 6. Each channel support 5 may be welded or otherwise fastened to the cabinet 4. Typically, the cabinet 4, any channel supports 5, and the frame 26 of the lower assembly 6 will be constructed of stainless steel or other suitable materials.

Appropriate closable access means, such as one or more cabinet doors 12 located opposite the back 80 of the cabinet 4, both provide access to the interior of the cabinet 4 and allow the cabinet 4 to be closed. Each cabinet door 12 may be attached to the side 7 of a cabinet 4 by way of one or more hinges 16 and secured in the closed position shown in FIG. 1 by way of one or more door latches 14 or other appropriate securing means. One may maintain each cabinet door 12 in the open position by way of one or more door clips 8 or other appropriate fastening means. Each cabinet door 12 may include a seal (not illustrated) for improved protection of the integrity of any contents of the cabinet 4 from contaminants. The cabinet 4 may be raised and lowered for the convenience of the user and improved ergonomics.

The lower assembly 6 may include one or more solid shelves 28, as well as one or more grated shelves 30. These may be used to transport any number of items that may not be required to be maintained in a sterilized state within the cabinet 4. The lower assembly 6 further comprises a plurality of wheels 20, 22 that facilitate the portability of the self-washing closable surgical case cart apparatus 2. As illustrated in this particular embodiment, fixed wheels 20 may be utilized on one end of the self-washing closable surgical case cart apparatus 2 and directional wheels 22 may be included on the other end to facilitate maneuverability. Castor brakes 24 provide a means of securing the self-washing closable surgical case cart apparatus 2 in a given stationary position. Handles 18 on either end of the cabinet 4 further assist a user in transporting and maneuvering the self-washing closable surgical case cart apparatus 2.

One or more inlet couplers 32 in the cabinet 4 allows a decontaminating fluid source to be introduced into the cabinet 4 by attaching a hose or other suitable fluid transmission channel. Each inlet coupler 32 leads to one or more fluid supply lines 33 within the cabinet 4, which in turn leads to a ball tank washer 34 or other appropriate fluid distribution means, also within the cabinet 4. The ball tank washer or other fluid distribution means may be located within the interior portion of the cabinet 4 under the roof 10 or in any other suitable location to enable 360-degree dispersal of fluids throughout the interior of the cabinet 4.

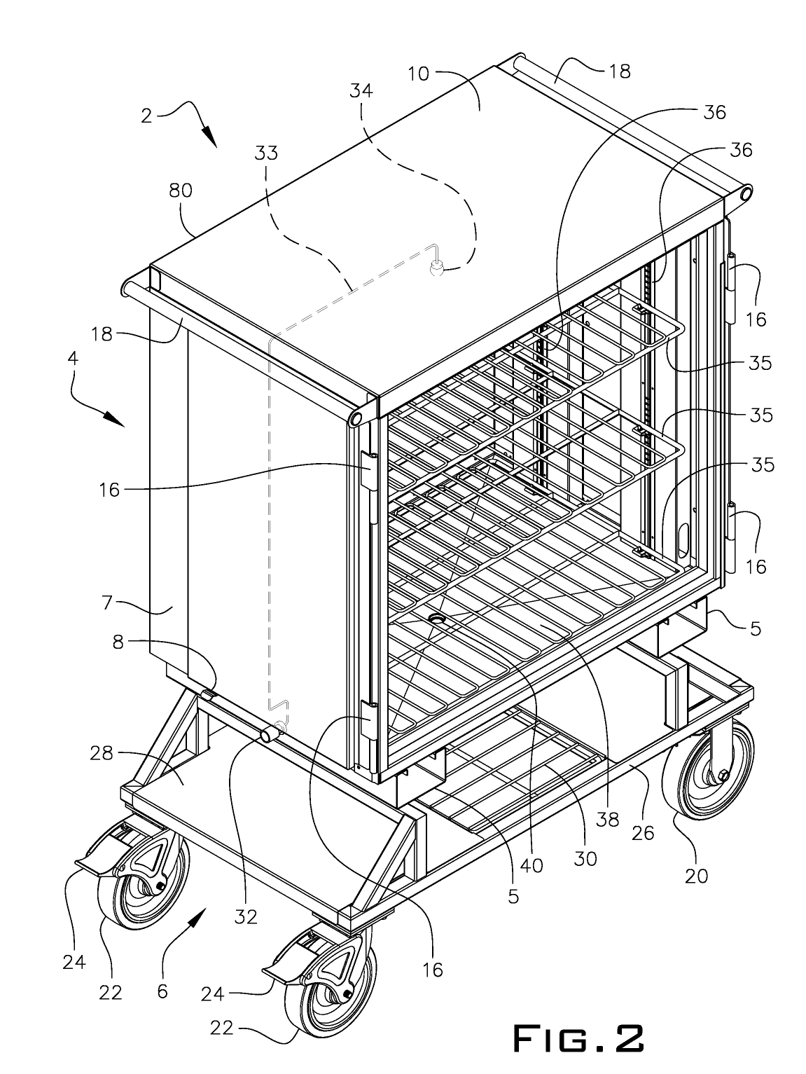

In FIG. 2, the doors 12 have been rotated about the hinges 16 to an open position and secured against the sides 7 of the cabinet 4 by means of door clips 8, revealing the interior of the cabinet 4. As illustrated, one or more internal shelves 35 may be adjustably positioned within the cabinet 4 by means of adjustable shelf supports 36, which may be located on the interior portion of either of side 7 of the cabinet 4. A pitched floor 38 may be incorporated into the cabinet 4 to direct gravitationally any fluids towards one or more pluggable drains 40 in or about said pitched floor 38.

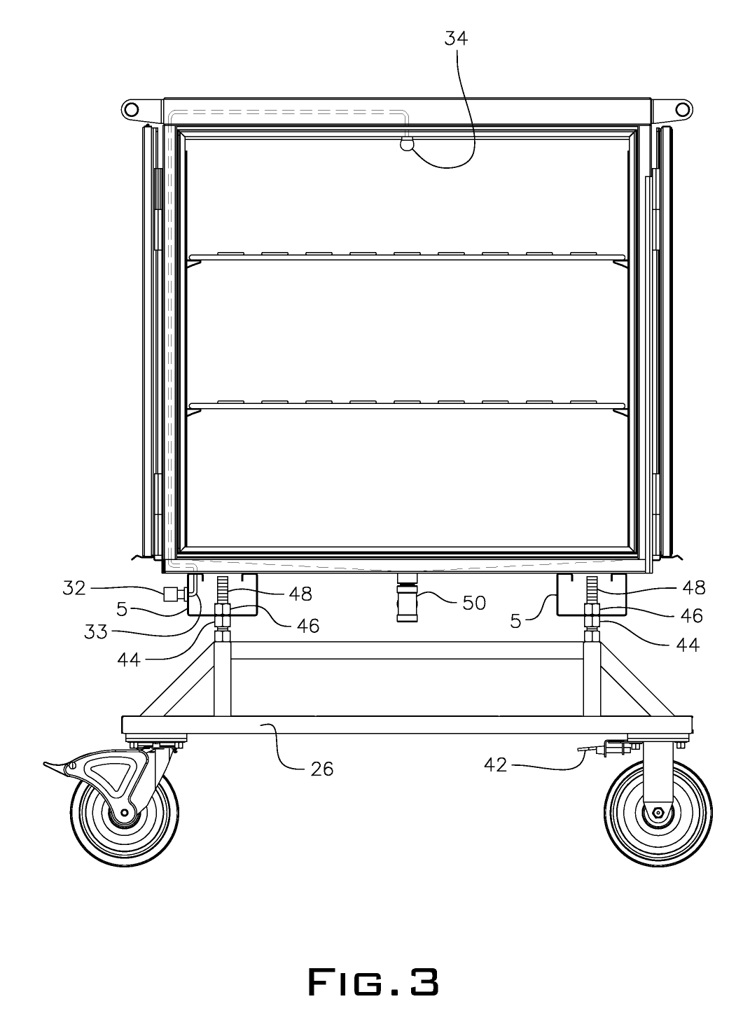

In the front view of the cabinet 4 in an open state shown in FIG. 3, the cabinet 4 is shown adjusted to its lowest position. This is accomplished by means of a threaded shaft 48 extending from the frame 26 of the lower assembly 4 through each channel support 5, an upper nut 46 and a lower nut 44. In combination, a channel support 5, such a threaded shaft 48, upper nut 46 and lower nut 44 may be referred to as a case leveler. In the lowest position, an interior shelf 35 at the lowest vertical point along the adjustable shelf supports 36 within the cabinet 4 must be no less than 18 to 24 inches above any surface on which the self-washing closable surgical case cart apparatus 2 rests.

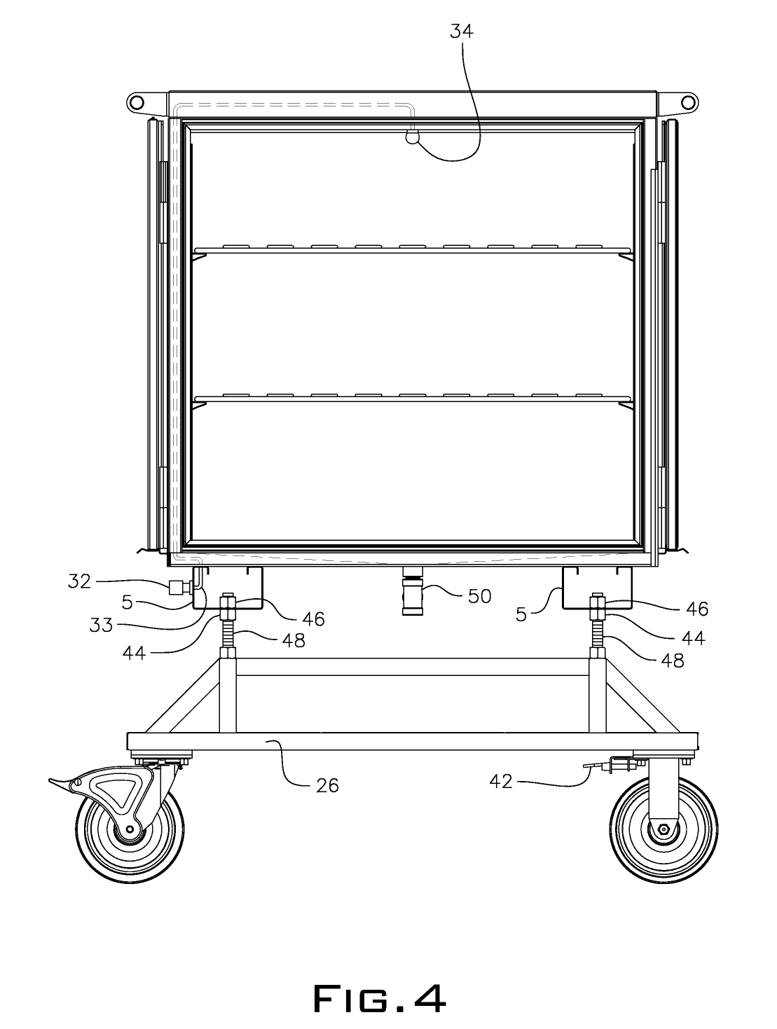

One or more outlet couplers 50, one of which is illustrated in FIG. 3, may be included to allow the attachment of a hose or other suitable fluid drainage channel to transmit waste fluid from the one or more pluggable drains 40 in lieu of allowing free drainage. A wheel lock 42, used to secure one or more fixed wheels 20 into a stationary position, may also be seen. FIG. 4 illustrates the cabinet 4 adjusted to its highest position.

The design of the instant invention provides improved ergonomics for users as well as enhanced protection from floor-level contaminants. As discussed, the lowest position for a shelf within the self-washing closable surgical case cart apparatus 2 is positioned a minimum of 18 to 24 inches above the floor, reducing bending and lifting when loading and unloading, and creating greater distance from potential contamination from the floor. The wheels 20, 22 of the self-washing closable surgical case cart apparatus 2 may be larger than those commonly used in other surgical case cart designs to allow for easier maneuverability, and directional wheels 22 aid in steering while the self-washing closable surgical case cart apparatus 2 is in transit.

Methods of decontaminating the self-washing closable surgical case cart apparatus 2, taking advantage of its unique self-washing features, are also disclosed. Broadly, decontamination includes the steps of connecting a decontaminating fluid source to the self-washing closable surgical case cart apparatus 2 via suitable connection means, introducing the decontamination fluid or fluids into the self-washing closable surgical case cart apparatus 2 while in a closed state, disconnecting the decontaminating fluid source, and allowing the self-washing closable surgical case cart apparatus 2 to air dry or towel-drying it prior to placing it into service.

In one particular embodiment of a decontamination method, a water source is first established for a wash-cycle control monitor with a cycle switch and a monitor reservoir. This source may be either plumbed permanently to the wash-cycle control monitor or accessed via a hot water faucet through a hose or other appropriate fluid transmission channel.

Once the water supply is thus connected to the wash-cycle control monitor—which may be mounted to a wall or other appropriate location—a concentrated enzyme detergent of choice may be added to the monitor reservoir. The wash-cycle control monitor is then attached via a hose or other appropriate fluid transmission channel via an inlet coupler 32 to the cabinet 4 of a self-washing closable surgical case cart apparatus 2 in a closed state. Any desired hose or other suitable fluid drainage channel may be attached to the cabinet 4 via an outlet coupler 50 and directed to a receptacle or other receiving point for waste fluid from the interior of the cabinet 4.

Next, the water source is activated, and the cycle switch on the wash-cycle control monitor activated to deliver the concentrated enzyme detergent into the water from the water source. Either a single or a double enzyme wash of the cabinet 4 may be carried out at this point.

A rinse cycle may then be undertaken with plain water to eliminate any residual enzyme detergent from the interior of the cabinet 4. A second rise cycle optionally may follow.

Finally, the hose or other appropriate fluid transmission channel is disconnected from the inlet coupler. Following air-drying or towel-drying of the interior of the cabinet 4, the self-washing closable surgical case cart apparatus 2 may be placed into service.

What is claimed is:

1. A self-washing closed surgical case cart apparatus, comprising:
a cabinet;
a lower assembly;
closable access means;
securing means;
one or more inlet couplers;
one or more fluid supply lines;
fluid distribution means; and
one or more pluggable drains.

2. The self-washing closable surgical case cart apparatus of claim 1, in which the closable access means are doors, and the securing means are latches.

3. The self-washing closable surgical case cart apparatus of claim 2, further comprising fastening means.

4. The self-washing closable surgical case cart apparatus of claim 3, in which the fastening means are door clips.

5. The self-washing closable surgical case cart apparatus of claim 2, further comprising seals.

6. The self-washing closable surgical case cart apparatus of claim 1, further comprising one or more case levelers.

7. The self-washing closable surgical case cart apparatus of claim 1, further comprising one or more internal shelves and adjustable shelf supports.

8. The self-washing closable surgical case cart apparatus of claim 6, in which the lowest position for a shelf within the self-washing closable surgical case cart apparatus when said self-washing closable surgical case cart apparatus has been adjusted to its lowest position using said one or more case levelers is a minimum of 18 inches above the floor.

9. The self-washing closable surgical case cart apparatus of claim 1, further comprising one or more outlet couplers.

10. The self-washing closable surgical case cart apparatus of claim 1, in

11. The self-washing closable surgical case cart apparatus of claim 10, further comprising one or more castor brakes and one or more wheel locks.

12. The self-washing closable surgical case cart apparatus of claim 1, further comprising one or more handles.

13. A self-washing closed surgical case cart system, comprised of:
A self-washing closed surgical case cart apparatus;
A decontaminating fluid source; and
Connection means to supply said decontaminating fluid source to said self-washing closed surgical case cart apparatus.

14. The self-washing closed surgical case cart system of claim 13, in which the decontaminating fluid source comprises a water source.

15. The self-washing closed surgical case cart system of claim 13, in which the connection means comprise one or more fluid transmission channels and a wash-cycle control monitor with a cycle switch and a monitor reservoir by way of which a concentrated enzyme detergent or other agent may be introduced into a decontaminating fluid source.

16. The self-washing closed surgical case cart system of claim 13, further comprising one or more fluid drainage channels.

17. A method of decontaminating a self-washing closable surgical case cart, comprising the steps of:
Connecting a decontaminating fluid source to a self-washing closable surgical case cart apparatus in a closed state via connection means;
Introducing one or more decontamination fluids into said self-washing closable surgical case cart apparatus;
Disconnecting said decontaminating fluid source from the self-washing closable surgical case cart apparatus;
Opening said self-washing closable surgical case cart apparatus; and
Allowing said self-washing closable surgical case cart apparatus to air dry or towel-drying it.

18. A method of decontaminating a self-washing surgical case cart, comprising the steps of:
Connecting a water source via a first fluid transmission channel to a wash-cycle control monitor with a cycle switch and a monitor reservoir capable of accepting a concentrated enzyme detergent or other agent for introduction into water from said water source;
Adding a concentrated enzyme detergent to said monitor reservoir;
Connecting said wash-cycle control monitor via a second fluid transmission channel to a self-washing surgical case cart in a closed state;
Activating said water source;
Switching on said cycle switch to cause the concentrated enzyme detergent in the monitor reservoir to be introduced into water from the water source prior to the delivery of the water to the self-washing surgical case cart until all the detergent in the monitor reservoir has been exhausted;
Continuing to allow the water from the water source to flow into the self-washing closed surgical case cart apparatus so as to rinse any remaining enzyme detergent from the interior of the self-washing closed surgical case cart apparatus;
Disconnecting the second fluid transmission channel from the self-washing closed surgical case cart apparatus;
Opening said self-washing closable surgical case cart apparatus; and
Air-drying or towel drying of the interior of said self-washing closable surgical case cart apparatus.
